(19) United States
(12) Patent Application Publication
(10) Pub. No.: US 2003/0195571 A1
(43) Pub. Date: Oct. 16, 2003

(54) Method and apparatus for the treatment of central sleep apnea using biventricular pacing

(76) Inventors: John E. Burnes, Andover, MN (US); Yong K. Cho, Maple Grove, MN (US)

Correspondence Address: MEDTRONIC, INC., 710 MEDTRONIC PARKWAY NE, MS-LC340, MINNEAPOLIS, MN, 55432-5604, US

(21) Appl. No.: 10/121,323

(22) Filed: Apr. 12, 2002

(51) Int. Cl. A61N 1/36

(52) U.S. Cl. 607/9

(57) ABSTRACT

An apparatus and method for treating sleep apnea includes a control unit in electrical communication with a lead. The control unit is capable of outputting a sleep apnea interruption pulse to stimulate at least one of a phrenic nerve and a diaphragm. Specifically, an implanted medical device (IMD) such as an ICD or a pacemaker paces the heart and a mode switch algorithm changes the pacing output to stimulate at least one of a phrenic nerve and diaphragm when sleep apnea is detected by the control unit. The method includes determining if the patient is experiencing sleep apnea and outputting a sleep apnea interruption pulse to the at least one of a phrenic nerve and a diaphragm. The control unit may be incorporated with the IMD. In another embodiment, the control unit may be in wireless communication with the IMD and positioned outside a patient's body.

### FIELD OF THE INVENTION

The present invention generally relates to implantable medical devices. Specifically, the invention relates to the prevention of hypopnia during sleep apnea by stimulating the phrenic nerve with implanted cardiac leads, when the onset of sleep apnea is detected. More specifically, the invention relates to a biventricular pacemaker adapted to provide an automatically adjustable output via a lead preferably located in the coronary sinus.

### BACKGROUND OF THE INVENTION

Sleep apnea is generally associated with the cessation of breathing during sleep. The medical characteristics of sleep apnea have been known for some time. Sleep apnea is terminated by the subject's arousal, followed by hyperventilation. Such arousals from sleep are generally associated with increased sympathetic nervous system activity and blood pressure, which may contribute to the worsening of a patient's cardiac condition.

Generally, there are two types of sleep apnea. The first is central sleep apnea, which relates to the failure of the body to automatically generate the neuro-muscular stimulation necessary to initiate and control the respiratory cycle at the proper time. The second sleep apnea syndrome is known as obstructive sleep apnea. This generally relates to an obstructive apnea that includes reduction of the size of the superior airways, an increase in their compliance and reduction in the activity of the dilator muscles.

In the prior art, there are disclosures that suggest various methods and structures to treat events of sleep apnea. For example, in U.S. Pat. No. 6,126,611 to Bourgeois et al., a system is disclosed for stimulating the heart at a higher rate than the heart's natural rate when an apnea event is detected.

Further examples of pertinent prior art include: U.S. Pat. No. 6,091,973 to Colla et al. discloses a diagnostic system for determining an apneic or hypopneic arousal; U.S. Pat. No. 5,974,340 to Kadhiresan discloses apparatus and method for monitoring respiratory function in heart failure patients to determine the efficacy of therapy; U.S. Pat. No. 5,591,216 to Testerman et al. discloses a method for opening an upper airway of a patient by applying electrical stimulation to the patient's hypoglossal nerve; and U.S. Pat. No. 5,540,733 to Testerman et al. discloses a method and apparatus for detecting and treating obstructive sleep apnea. Electrical stimulation of muscles of the upper airway, including detection of obstructive apnea and stimulation of the muscles of the upper airway in response to the apnea is disclosed.

Moreover, U.S. Pat. No. 5,540,732 to Testerman discloses method and apparatus for impedance detecting and treating obstructive airway disorders. In this disclosure, an implanted impedance-sensing circuit provides a signal characteristic of transthoracic impedance in the patient. The implanted impedance-sensing circuit allows the inspiratory phase of the patient's respiratory cycle to be identified to apply electrical stimulation during the inspiration phase.

U.S. Pat. No. 5,540,731 to Testerman discloses a method and apparatus for pressure detection and treating obstructive airway disorders. In this disclosure, muscles of the upper airway are stimulated based on a signal acquired from a pressure sensor thus implanted in the patient. The signal is characteristic of intrathoracic pressure in the patient. The pressure sensor enables the identification of the patient's respiratory cycle, such that the electrical stimulation could be applied during the inspiration phase.

U.S. Pat. No. 5,483,969 discloses a method and apparatus for providing a respiratory effort waveform for the treatment of an obstructive sleep apnea. In this disclosure, a digital respiratory effort waveform is used to stimulate an upper airway muscle of a patient. Specifically, the waveform is provided by sensing a signal having an output characteristic of respiratory effort of the patient and sampling the sense signal at the predetermined interval.

Further, U.S. Pat. No. 5,335,657 to Terry Jr. et al. discloses a nervous stimulation system to treat sleep disorder. Specifically, sleep disorder is detected and a predetermined electrical signal to the patient's vegus nerve is applied to alleviate the sleep disorder. The disclosure also relates to sensing the patient's ECG activity in the case of insomniac and hypersomniac patients or detecting a sudden nodding of the head in the case of narcoleptic patients, or sensing the cessation of respiration in the case of sleep apnea patients.

U.S. Pat. No. 5,146,918 to Kallok et al. discloses a demand apnea control of central and obstructive sleep apnea. The disclosure relates to the use of electrical stimulation on a demand basis. Specifically, sensors monitor the respiration cycle and determine the occurrence of apnea events. More specifically, central apnea is sensed by the passage of an escape interval of time, without the sensing of an aspiratory event and a concurrent decrease in blood oxygen saturation. Obstructive sleep apnea is sensed as an abnormal pressure differential across the airway. The diaphragm is electrically stimulated upon sensing of central apnea and if obstructive sleep apnea is detected, the musculature of the upper airway is electrically stimulated.

Accordingly, prior art systems typically manage sleep apnea by implanting electrodes in sensors to stimulate the diaphragm and/or musculature of the upper airway. However, most of these apparatus and methods involve complicated implant procedures and appear to be highly invasive. The present invention provides a novel approach that eliminates these complications and the various limitations of the prior art.

### SUMMARY OF THE INVENTION

In one aspect of the present invention, an apparatus for treating sleep apnea is presented. The apparatus includes a control unit and a lead extending from the control unit and having an electrode electrically coupled with the control unit by a conductor, the lead being capable of being implanted proximate a blood-carrying structure within a patient's body. The control unit is capable of outputting a sleep apnea interruption pulse via the conductor and the electrode to stimulate at least one of a phrenic nerve and a diaphragm.

In another aspect of the present invention, a method for treating sleep apnea is presented. The method includes determining if the patient is experiencing sleep apnea and outputting a sleep apnea interruption pulse to at least one of a phrenic nerve and a diaphragm if the patient is experiencing sleep apnea.

Yet another aspect of the present invention includes an implanted medical device that delivers therapy to interrupt sleep apnea in conjunction with cardiac therapy that is being delivered. Specifically, a mode switch algorithm changes pacing outputs of a pacemaker, a cardioverter or cardioverter defibrillator. More specifically, a phrenic nerve stimulation threshold is set such that by increasing the pulse widths or increasing the amplitude, or both, phrenic nerve stimulation and cardiac stimulation can be maintained.

In a further aspect of the present invention, rather than using bipolar leads or stimulating from the LV lead to the RV lead, it is suggested to stimulate from the LV lead to the can. This changes the field that may capture the phrenic nerve. Accordingly, a pacing configuration that captures the phrenic nerve is set in combination with or coordinated with a cardiac pacing scheme. Specifically, when sleep apnea is detected and the need to interrupt it is confirmed, the pacing scheme may be switched to operate under a one pulse delivery made such that both the heart and the phrenic nerve are stimulated. Subsequently, the pacing configuration is switched back to a normal pacing of the heart.

In yet another aspect of the invention, a pacemaker is implemented having a second mode that allows to pace the phrenic nerve and/or the diaphragm. When sleep apnea is sensed, stimulation of the phrenic nerve may be implemented by either changing the pacing configuration, increasing amplitudes, changing the pacing, changing the electrodes that are used to pace between, changing the number of pulses that are generated by the pacemaker, or implementing a train of pulses rather than a single pulse.

Yet another aspect of the present invention includes synchronization of the phrenic nerve stimulation pacing with therapeutic pacing that is due to be delivered to the heart. In yet another alternate embodiment, synchronization with the intrinsic heart rate is implemented to trigger phrenic nerve stimulation off a sensed beat.

### BRIEF DESCRIPTION OF THE DRAWINGS

The invention may be understood by reference to the following description taken in conjunction with the accompanying drawings, in which the leftmost significant digit(s) in the reference numerals denote(s) the first figure in which the respective reference numerals appear, and in which:

FIG. 1 is a stylized view of an embodiment of an implantable medical device according to the present invention for use in treating sleep apnea;

FIG. 2 is a stylized view of an implantable medical device lead according to the present invention that is attached to a myocardium of a heart for use in treating sleep apnea;

FIG. 3 is a stylized view of a lead according to the present invention having a partial ring electrode for use in treating sleep apnea;

FIG. 4 is a stylized view of a lead according to the present invention disposed within vasculature proximate a phrenic nerve and a diaphragm;

FIG. 5 is a flowchart of a first embodiment of a method according to the present invention for treating sleep apnea; and

FIG. 6 is a flowchart of a second embodiment of a method according to the present invention for pacing a rhythm of a heart and for treating sleep apnea.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF SPECIFIC EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

The present invention encompasses an apparatus and method for managing sleep apnea by stimulating a patient's phrenic nerve and/or diaphragm through the use of one or more electrodes disposed in a patient's body, such as the patient's heart, vasculature, or the like. The one or more electrodes may be placed proximately within the blood-carrying structure or proximately outside the blood-carrying structure. The phrenic nerve includes branches from the C3 through C5 spinal nerves and descends therefrom, through the thorax proximate to the heart, to the diaphragm. Electrical signals are transmitted through the phrenic nerve from the brain to cause the diaphragm to move, thus producing respiration.

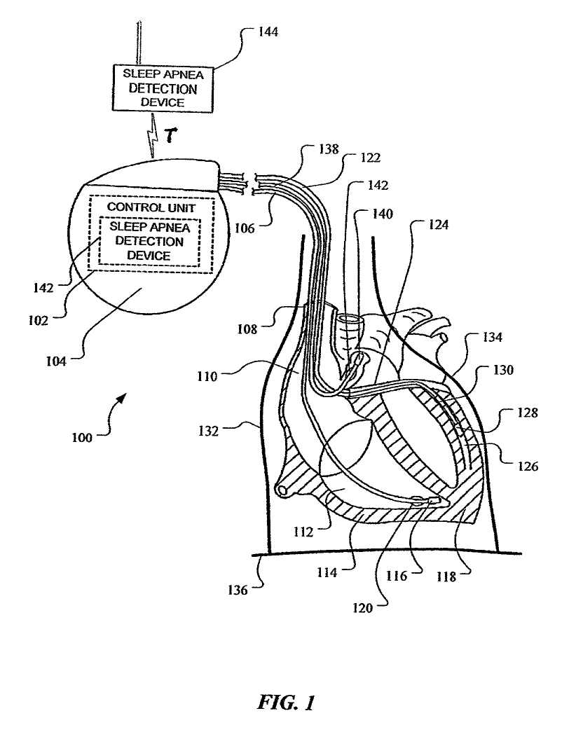

FIG. 1 illustrates an implantable medical device 100 according to the present invention including a control unit 102 enclosed in a biocompatible, hermetically sealed can 104. The implantable medical device 100 further includes a first lead 106 extending from the control unit 102, which may be routed through a superior vena cava 108, a right atrium 110, and into a right ventricle 112 of a heart 114. The first lead 106 includes a tip electrode 116 that may be disposed proximate an apex 118 of the heart 114 and a ring electrode 120 that may be disposed within the right ventricle 112 of the heart 114. While FIG. 1 illustrates the tip electrode 116 disposed proximate the apex 118 of the heart 114, the tip electrode 116 may be disposed anywhere within the right ventricle 112 of the heart 114.

The implantable medical device 100 also includes a second lead 122 extending from the control unit 102, which may be routed through the superior vena cava 108, the right atrium 110, a coronary sinus 124, and into a cardiac vein 126 (e.g., a middle cardiac vein, a great cardiac vein, or the like). The second lead 122 includes a tip electrode 128 and a ring electrode 130. The tip electrode 128 is generally disposed distally within the cardiac vein 126 from the ring electrode 130 with respect to the control unit 102.

The implantable medical device 100 may further include, as illustrated in FIG. 1, a third lead 138 extending from the control unit 102, which may be routed through the superior vena cava 108 and into the right atrium 110. The third lead 138 includes a tip electrode 140 and a ring electrode 142. The tip electrode 140 is generally disposed distally from the ring electrode 142 with respect to the control unit 102. The leads 106, 122, 138 may be unipolar or multipolar, thus having any number of electrodes (e.g., the electrodes 116, 120, 128, 130, 140, 142, or the like) as desired.

Generally, electrical pulses may be outputted from the control unit 102, via the leads 106, 122, 138 to one or more of the electrodes 116, 120, 128, 130, 140, 142 so that a portion of body tissue (e.g., a portion of the heart 114, a nerve or nerve bundle, a diaphragm 136, or the like) may be stimulated. For example, electrical pulses may be outputted from the control unit 102 via the first lead 106 to the tip electrode 116 of the first lead 106, wherein the electrical pulses may be useful in stimulating the right ventricle 112 of the heart 114. The electrical circuit is completed, in this example, by returning at least a portion of the electrical energy comprising the pulses via the ring electrode 120 of the first lead 106 and the lead 106 to the control unit 102.

In another example, electrical pulses may be outputted from the control unit 102 via the first lead 106 to the tip electrode 116 of the first lead 106, wherein the electrical circuit is completed by returning at least a portion of the electrical energy comprising the pulses via the can 104 to the control unit 102. Other configurations and modes of operation may be employed, such that electrical pulses are emitted from certain ones of the electrodes 116, 120, 128, 130, 140, 142 and returned to the control unit 102 via other ones of the electrodes 116, 120, 128, 130, 140, 142 and/or the can 104.

Still referring to FIG. 1, a right phrenic nerve 132 extends proximate a right side of the heart 114 and a left phrenic nerve 134 extends proximate a left side of the heart 114. As discussed above, each of the right phrenic nerve 132 and the left phrenic nerve 134 extends to the diaphragm 136. It has been found that, under certain circumstances, electrical pulses emitted from electrodes (e.g., the electrodes 116, 120, 128, 130, 140, 142, or the like) disposed within and/or proximate blood-carrying structures, such as the heart 114, vasculature, or the like, may stimulate one or both of the right phrenic nerve 132 and the left phrenic nerve 134. Such stimulation may result in stimulation of the diaphragm 136. Further, the electrical pulses may stimulate the diaphragm 136 directly. Accordingly, the scope of the present invention encompasses the direct stimulation of the diaphragm 136 by such electrical pulses as well as stimulation of the diaphragm 136 via the phrenic nerves 132, 134. Thus, according to the present invention, one or more electrical pulses may be outputted from the control unit 102, transmitted via one or more of the leads 106, 122, 138, and emitted from one or more electrodes (e.g., the electrodes 116, 120, 128, 130, 140, 142 or the like) disposed proximate a blood-carrying structure to stimulate one or both of the phrenic nerves 132, 134 to stimulate the diaphragm 136.

The control unit 102 may, in one embodiment, also include a sleep apnea detection device 142 for determining whether the patient is experiencing sleep apnea. Sleep apnea detection device 142 may be incorporated with implantable medical device (IMD) or can 104. In an alternate embodiment, sleep apnea detection device 142 is in wireless/telemetry communication T with can 104. The sleep apnea detection device 142, 144 may operate by any means known in the art. For example, sleep apnea may be detected by cycle breath analysis, heart rate variability, bradycardia sensing, minute ventilation sensing; pressure/impedance sensing, inspiratory function sensing, diaphragm contraction sensing, airflow sensing via nostrils or a mouth, and/or the like.

Those skilled in the art will appreciate that electrical pulses are conventionally used to pace one or more chambers (e.g., the right ventricle 112, the right atrium 110, or the like) of the heart 114. Generally, such electrical pulses are effective only on the portion of the heart proximate to the electrode or electrode from which the electrical pulses are being emitted, due to the amplitude, shape, and/or duration of the pulses. Conventionally, this is generally a desirable situation, since it may be undesirable to stimulate other body tissue proximate the heart 114. However, in the treatment of sleep apnea, it may be generally desirable, according to the present invention, to stimulate one or both of the phrenic nerves 132, 134 and/or the diaphragm 136, either alone or in combination with a portion of the heart 114. Thus, according to one embodiment of the present invention, the amplitude and/or duration of the pulses is modified from the pulses generally used in cardiac pacing therapies to stimulate one or both of the phrenic nerves 132, 134 and/or the diaphragm 136 and, in certain circumstances, a portion of the heart 114.

For example, in conventional cardiac pacing therapies, the amplitude of the electrical pulses may fall within a range of about 0.5V to about 5.0V. By comparison, the amplitude of the electrical pulses useful in stimulating one or both of the phrenic nerves 132, 134 and/or the diaphragm 136, according to the present invention, may fall within a range of about 0.5V to about 10V. Thus, when stimulating one or both of the phrenic nerves 132, 134 and/or the diaphragm 136, the portion or portions of the heart 114 proximate the electrode or electrodes being used to stimulate the phrenic nerves 132, 134 may also be stimulated. Accordingly, according to one embodiment of the present invention, the electrical pulses used to stimulate the phrenic nerves 132, 134 are timed to coincide with a desirable time for stimulating the portion or portions of the heart 114 proximate the electrode or electrodes being used to stimulate the phrenic nerves 132, 134. For example, the electrical pulses may be timed to coincide with an intrinsic heartbeat, a planned cardiac pacing pulse, or may be timed based on a previous intrinsic heartbeat or cardiac pacing pulse. In this way, normal cardiac function may be maintained without inducing arrhythmia in the heart 114.

Further, in conventional cardiac pacing therapies, the duration of the electrical pulse may generally fall within a range of about 0.05 ms to about 0.5 ms. However, to effectively stimulate one or both of the phrenic nerves 132, 134 and/or the diaphragm 136, according to the present invention, the duration of the electrical pulse may fall within a range of about 0.5 ms to about 1.5 ms. As described above, such electrical pulses used to stimulate one or both of the phrenic nerves 132, 134 and/or the diaphragm 136 may also stimulate the portion or portions of the heart 114 proximate the electrode or electrodes being used to stimulate the phrenic nerves 132, 134. Thus, it may be desirable, as described above, to time the stimulation of the phrenic nerves 132, 134 to coincide with a desirable point in time to stimulate the portion or portions of the heart 114 proximate the electrode or electrodes being used to stimulate the phrenic nerves 132, 134.

While specific voltage and duration ranges are provided above, the scope of the present invention encompasses any pulse voltage or duration, or any series of pulse voltages and durations, which are effective in stimulating one or both of the phrenic nerves 132, 134 and/or the diaphragm 136. The amplitude and/or duration of the electrical pulse required to stimulate the phrenic nerves 132, 134 and/or the diaphragm 136 may depend upon where the electrode is positioned relative to one of the phrenic nerves 132, 134 and/or the diaphragm 136. For example, in general, the more distally the electrode is positioned from the phrenic nerve 132, 134 and/or the diaphragm 136, the greater the pulse amplitude and/or the pulse duration required to stimulate the phrenic nerve 132, 134 and/or the diaphragm 136. Many features of the human anatomy, such as locations of the coronary veins, position of the phrenic nerves 132, 134 and/or the diaphragm 136 with respect to a blood-carrying structure, are quite variable from patient to patient. Thus, it may be desirable to position the electrode or electrodes and to determine the amplitude and/or duration of the pulse to be used to stimulate one or both of the phrenic nerves 132, 134 and/or the diaphragm 136 in an iterative fashion. For example, it may be desirable to position the electrode then output a pulse to determine if the phrenic nerve 132, 134 and/or the diaphragm 136 may be stimulated at that electrode position, pulse amplitude, and pulse duration. If stimulation is accomplished, the amplitude and/or duration of the pulse may be reduced to determine if the phrenic nerve 132, 134 and/or the diaphragm 136 may still be stimulated. If stimulation is not accomplished at the first electrode position, pulse amplitude setting, and pulse duration setting, the electrode may be repositioned or the pulse amplitude and/or pulse duration may be increased to determine if the phrenic nerve 132, 134 and/or the diaphragm 136 may be stimulated.

In certain circumstances it may be possible to directly stimulate the diaphragm 136 by emitting stimulation pulses from an electrode disposed proximate a blood-carrying structure within a patient's body. For example, the tip electrode 116 of the first lead 106 may be disposed close enough to the diaphragm 136 such that stimulation pulses emitted from the tip electrode 116 may capture the diaphragm 136. Thus, such pulses may stimulate the diaphragm 136 directly with little or no interaction with the phrenic nerves 132, 134.

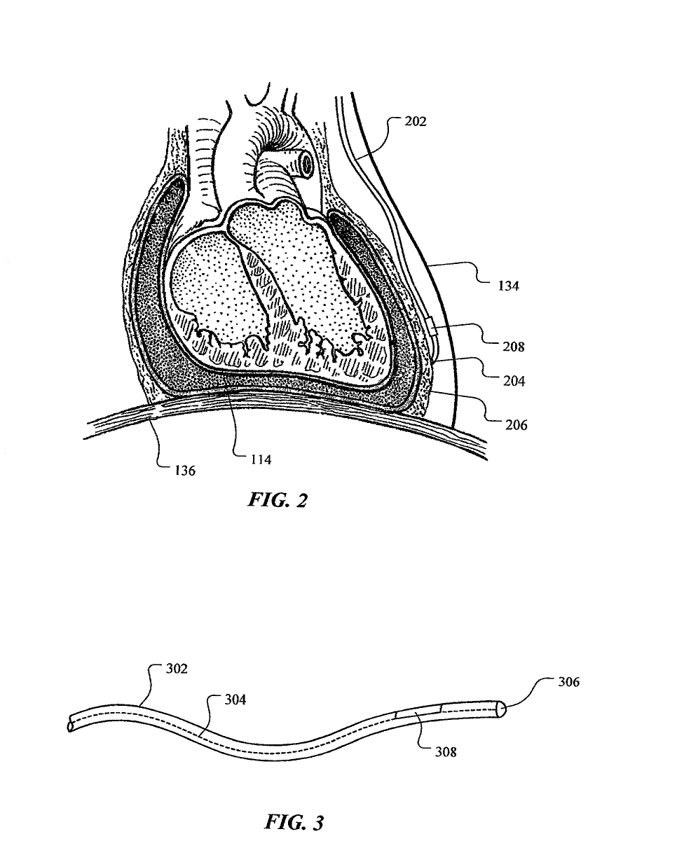

While a plurality of leads 106, 122, 138 are illustrated in FIG. 1, the present invention encompasses an implantable medical device 100 having only one of the leads 106, 122, 138. Further, the scope of the present invention includes an implantable medical device 100 having one or more leads (e.g., the leads 106, 122, 138, or the like) extending from the control unit 112 to areas proximate blood-carrying structures other than as shown in FIG. 1. For example, the present invention encompasses an implantable medical device 100 having a lead 202, as illustrated in FIG. 2, having a tip electrode 204 and extending from the control unit 102 (shown in FIG. 1) to a pericardium 206 of the heart 114. The phrenic nerve 134 and/or the diaphragm 136 may be stimulated either via the tip electrode 204 or via an optional ring electrode 206.

Further, it may be possible to stimulate one or both of the phrenic nerves 132, 134 and/or the diaphragm 136 while reducing the likelihood of stimulating the heart 114. FIG. 3 illustrates a lead 302 that may be used for either of the leads 122, 202 shown in FIGS. 1 and 2, respectively, and the like. The lead 302 includes a conductor set 304 having one or more conductors extending from the control unit 102 (shown in FIG. 1) to a tip 5 electrode 306 and a partial ring electrode 308. The partial ring electrode 308 extends only partway around a circumference of the lead 302. Thus, the lead 302 may be positioned proximate the heart 114 or within the cardiac vein 126 or the like such that the partial ring electrode 308 faces away from the heart 114. Accordingly, upon emitting a stimulation pulse from the partial ring electrode 308, the pulse is directed away from the heart 114, which may reduce the likelihood of stimulating a portion of the heart 114 proximate the partial ring electrode 308.

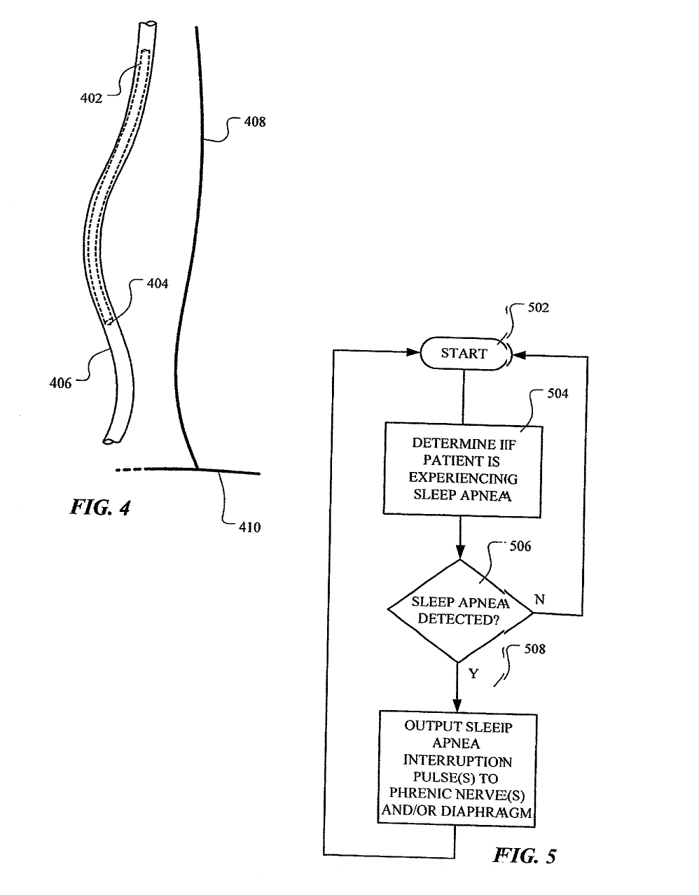

As indicated above, the scope of the present invention encompasses an electrode disposed within vasculature that is capable of stimulating one or more phrenic nerves and or the diaphragm of the patient. FIG. 4 illustrates a lead 402 having an electrode 404 and being disposed within a blood vessel 406 proximate a phrenic nerve 408 such that an electrical pulse or pulses, emitted from the electrode 404, may stimulate the phrenic nerve 408. Further, in one embodiment, the electrode 404 may be disposed within the blood vessel 406 proximate a diaphragm 410 such that an electrical pulse or pulses, emitted from the electrode 404, may directly stimulate the diaphragm 410.

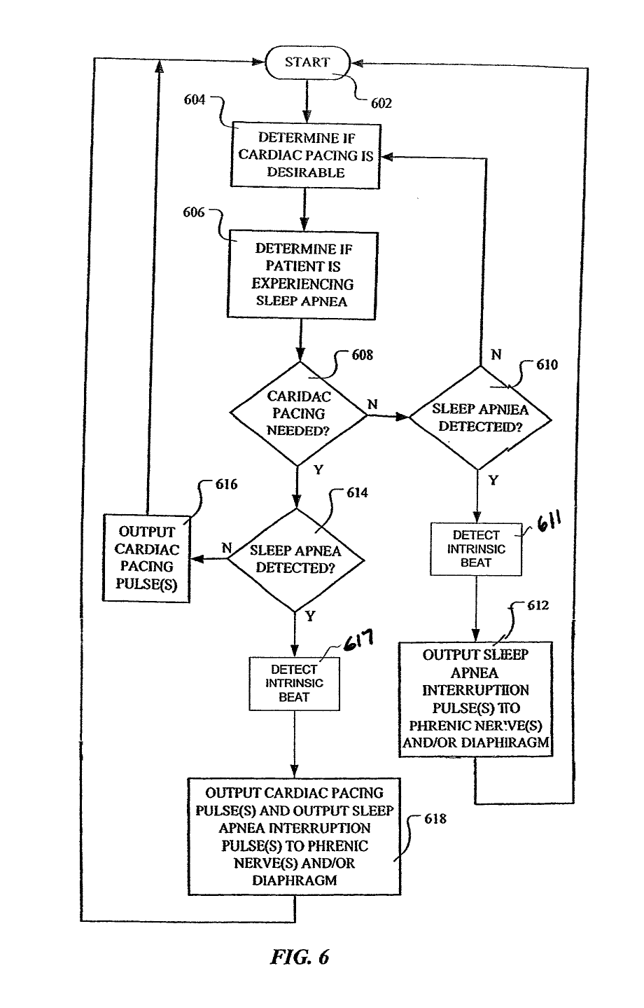

FIG. 5 illustrates a first embodiment of a method according to the present invention for treating sleep apnea. From a starting point (block 502), the method includes determining if the patient is experiencing sleep apnea (block 504). If the patient is not experiencing sleep apnea (block 506), the method returns to the starting point (block 502). If the patient is experiencing sleep apnea (block 506), however, the method includes outputting one or more sleep apnea interruption pulses to one or both of the phrenic nerves (e.g., the phrenic nerves 132, 134 shown in FIGS. 1 and 2) and/or to the diaphragm (e.g., the diaphragm 136 shown in FIGS. 1 and 2), as illustrated by block 508. In one embodiment, the one or more sleep apnea interruption pulses may be outputted (block 508) proximate a heart (e.g., the heart 114). In one embodiment, the pulses may be timed from a previously scheduled pacing pulse or triggered from an intrinsic beat of the heart 114, timed coincident with an intrinsic heartbeat, or timed to coincide with a cardiac pacing pulse. The method then returns to the starting point (block 508).

In certain situations, as described previously, it may be desirable to incorporate a method for treating sleep apnea into the pacing of a heart (e.g., the heart 114). Thus, a second embodiment of a method according to the present invention for treating sleep apnea, as illustrated in FIG. 6, includes, from a starting point 602, determining if cardiac pacing is desirable (block 604) and determining if the patient is experiencing sleep apnea (block 606). If cardiac pacing is not needed (block 608) and the patient is not experiencing sleep apnea (block 610), the method returns to the starting point (block 602). However, if the patient is experiencing sleep apnea (block 610), the method includes detection of intrinsic heart beat (block 611) and outputting one or more sleep apnea interruption pulses to one or both of the phrenic nerves (e.g., the phrenic nerves 132, 134 shown in FIGS. 1 and 2) and/or to the diaphragm (e.g., the diaphragm 136 shown in FIGS. 1 and 2), as illustrated by block 612. The method then returns to the starting point (block 602).

However, if cardiac pacing is needed (block 608) and the patient is not experiencing sleep apnea (block 614), the method includes outputting one or more cardiac pacing pulses (block 616). The method then returns to the starting point (block 602). If cardiac pacing is needed (block 608) and the patient is experiencing sleep apnea (block 614), the method includes detection of intrinsic heart beat (block 617) outputting one or more cardiac pacing pulses and outputting one or more sleep apnea interruption pulses to one or both of the phrenic nerves and/or the diaphragm (block 618). The method then returns to the starting point (block 602). In one embodiment, the one or more sleep apnea interruption pulses may be outputted (blocks 612, 618) proximate a heart (e.g., the heart 114).

While the method embodiment illustrated in FIG. 6 is described as having steps performed in a particular order, the present invention is not so limited. For example, determining if the patient is experiencing sleep apnea (block 606) may be performed prior to determining if cardiac pacing is desirable (block 604), or these steps may be performed simultaneously. Further, the decision of whether cardiac pacing is needed (block 608) may be performed after the decision of whether sleep apnea has been detected 5 (blocks 610, 614), or these steps may be performed simultaneously. Other variations of the method illustrated in FIG. 6 as will be appreciated to one skilled in the art are also encompassed by the present invention. Further, outputting the sleep apnea interruption pulses (blocks 612, 618) may also be used as the outputted cardiac pacing pulses (blocks 616, 618).

As indicated previously, the presence of a sleep apnea condition may be determined by any means known in the art. For example, sleep apnea may be detected by cycle breath analysis, heart rate variability, bradycardia sensing, minute ventilation sensing; pressure/impedance sensing, inspiratory function sensing, diaphragm contraction sensing, airflow sensing via nostrils or a mouth, and/or the like. Further, the desirability of cardiac pacing may be determined by any means known in the art, such as by analyzing one or more electrocardiograms, or the like. Thus, the specific means by which sleep apnea is detected and the specific means by which the desirability of cardiac pacing is determined are not material to the practice of the invention.

The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope and spirit of the invention. In particular, every range of values (of the form, “from about a to about b,” or, equivalently, “from approximately a to b,” or, equivalently, “from approximately a-b”) disclosed herein is to be understood as referring to the power set (the set of all subsets) of the respective range of values, in the sense of George Cantor. Accordingly, the protection sought herein is as set forth in the claims below.

What is claimed is:

1. An apparatus for treating sleep apnea, comprising:
a control unit; and
a lead extending from the control unit and having an electrode electrically coupled with the control unit by a conductor, the lead being capable of being implanted proximate a blood-carrying structure within a patient's body,
wherein the control unit is capable of outputting a sleep apnea interruption pulse via the conductor and the electrode to stimulate at least one of a phrenic nerve and a diaphragm.

2. The apparatus, according to claim 1, wherein the lead is capable of being implanted proximate a heart.

3. The apparatus, according to claim 1, wherein the lead is capable of being implanted within a chamber of the heart.

4. The apparatus, according to claim 1, wherein the lead is capable of being implanted within vasculature of the patient's body.

5. The apparatus, according to claim 1, wherein the lead is capable of being implanted within a cardiac vein.

6. The apparatus, according to claim 1, wherein the lead is capable of being placed within vasculature within a patient's body proximate a phrenic nerve.

7. The apparatus, according to claim 1, wherein the lead is capable of being attached to a pericardium of the heart.

8. The apparatus, according to claim 1, wherein the electrode further comprises a partial ring electrode capable of emitting the pulse therefrom in a predetermined direction.

9. The apparatus, according to claim 1, wherein the control unit is further capable of outputting a cardiac pacing pulse via the conductor and the electrode to stimulate a portion of the heart.

10. The apparatus, according to claim 1, wherein the sleep apnea interruption pulse also paces a heart rhythm.

11. The apparatus, according to claim 1, wherein the sleep apnea interruption pulse has an amplitude within a range of about 0.5 volts to about 10 volts.

12. The apparatus, according to claim 1, wherein the sleep apnea interruption pulse has a duration within a range of about 0.5 milliseconds to about 1.5 milliseconds.

13. The apparatus, according to claim 1, further comprising a sleep apnea detection device.

14. The apparatus, according to claim 1, wherein the control unit further comprises a sleep apnea detection device.

15. The apparatus, according to claim 1, wherein the control unit is capable of outputting a series of sleep apnea interruption pulses to stimulate at least one of the phrenic nerve and the diaphragm.

16. The apparatus, according to claim 1, further comprising a can encasing the control unit, wherein the sleep apnea interruption pulse is transmitted between the lead electrode and the can.

17. The apparatus, according to claim 1, further comprising a can encasing the control unit, wherein the lead electrode is a ring electrode and the control unit is capable of transmitting the sleep apnea interruption pulse between the electrode and the can.

18. The apparatus, according to claim 1, further comprising a can encasing the control unit, wherein the lead electrode is a tip electrode and the control unit is capable of transmitting the sleep apnea interruption pulse between the tip electrode and the can.

19. The apparatus, according to claim 1, wherein:
the lead electrode is a tip electrode;
the lead further comprises a ring electrode; and
the control unit is capable of transmitting the sleep apnea interruption pulse between the tip electrode and the ring electrode.

20. The apparatus, according to claim 1, further comprising a second lead having a second electrode electrically coupled with the control unit by a second conductor, the second lead being capable of being implanted within a patient's body, wherein the control unit is capable of transmitting the sleep apnea interruption pulse between the first lead electrode and the second lead electrode.

21. An apparatus for treating sleep apnea, comprising:
means for outputting a sleep apnea interruption pulse; and
means for conducting the sleep apnea interruption pulse to at least one of a phrenic nerve and a diaphragm.

22. The apparatus, according to claim 21, wherein the means for outputting the sleep apnea interruption pulse further comprises a control unit.

23. The apparatus, according to claim 21, wherein the means for conducting the sleep apnea interruption pulse further comprises a lead extending from the means for outputting the sleep apnea interruption pulse and having an electrode electrically coupled with the means for outputting the sleep apnea interruption pulse by a conductor, the lead being capable of being implanted proximate a portion of a heart.

24. The apparatus, according to claim 21, further comprising means for detecting sleep apnea.

25. The apparatus, according to claim 24, wherein the means for detecting sleep apnea further comprises a sleep apnea detection device.

26. The apparatus, according to claim 24, wherein the means for detecting sleep apnea further comprises a sleep apnea detection device and wherein the means for outputting the sleep apnea interruption pulse further comprises a control unit comprising the sleep apnea detection device.

27. A method for treating sleep apnea in a patient, comprising:
determining if the patient is experiencing sleep apnea; and
outputting a sleep apnea interruption pulse to at least one of a phrenic nerve and a diaphragm if the patient is experiencing sleep apnea.

28. The method, according to claim 27, wherein outputting the sleep apnea interruption pulse further comprises outputting the sleep apnea interruption pulse coincident with an intrinsic heartbeat.

29. The method, according to claim 27, wherein outputting the sleep apnea interruption pulse further comprises outputting the sleep apnea interruption pulse coincident with a cardiac pacing pulse

30. The method, according to claim 27, wherein outputting the sleep apnea interruption pulse further comprises outputting the sleep apnea interruption pulse at a predetermined time from a previous cardiac pacing event.

31. The method, according to claim 27, wherein outputting the sleep apnea interruption pulse further comprises outputting the sleep apnea interruption pulse at a predetermined time from a previous intrinsic heartbeat.

32. The method, according to claim 27, wherein outputting the sleep apnea interruption pulse further comprises outputting the sleep apnea interruption pulse proximate a portion of a heart.

33. The method, according to claim 27, further comprising determining if cardiac pacing is desirable.

34. The method, according to claim 33, further comprising outputting a cardiac pacing pulse to a portion of the heart without stimulating at least one of the phrenic nerve and the diaphragm, wherein outputting the sleep apnea interruption pulse further comprises outputting the sleep apnea interruption pulse coincident with the cardiac pacing pulse.

35. The method, according to claim 33, further comprising outputting a cardiac pacing pulse to pace a portion of a heart without stimulating at least one of the phrenic nerve and the diaphragm.

36. The method, according to claim 33, wherein outputting the sleep apnea interruption pulse further comprises outputting the sleep apnea interruption pulse to at least one of the phrenic nerve and the diaphragm and to a portion of a heart to pace a rhythm of the heart.

37. An apparatus for treating sleep apnea in a patient, comprising:
means for determining if the patient is experiencing sleep apnea; and
means for outputting a sleep apnea interruption pulse to at least one of a phrenic nerve and a diaphragm if the patient is experiencing sleep apnea.

38. The apparatus, according to claim 37, wherein the means for outputting the sleep apnea interruption pulse further comprises means for outputting the sleep apnea interruption pulse coincident with an intrinsic heartbeat.

39. The apparatus, according to claim 37, wherein the means for outputting the sleep apnea interruption pulse further comprises means for outputting the sleep apnea interruption pulse coincident with a cardiac pacing pulse

40. The apparatus, according to claim 37, wherein the means for outputting the sleep apnea interruption pulse further comprises means for outputting the sleep apnea interruption pulse at a predetermined time from a previous cardiac pacing event.

41. The apparatus, according to claim 37, wherein the means for outputting the sleep apnea interruption pulse further comprises means for outputting the sleep apnea interruption pulse at a predetermined time from a previous intrinsic heartbeat.

42. The apparatus, according to claim 37, wherein the means for outputting the sleep apnea interruption pulse further comprises means for outputting the sleep apnea interruption pulse proximate a portion of a heart.

43. The apparatus, according to claim 37, further comprising means for determining if cardiac pacing is desirable.

44. The apparatus, according to claim 43, further comprising means for outputting a cardiac pacing pulse to a portion of the heart without stimulating at least one of the phrenic nerve and the diaphragm, wherein the means for outputting the sleep apnea interruption pulse further comprises means for outputting the sleep apnea interruption pulse coincident with the cardiac pacing pulse.

45. The apparatus, according to claim 43, further comprising means for outputting a cardiac pacing pulse to pace a portion of a heart without stimulating at least one of the phrenic nerve and the diaphragm.

46. The apparatus, according to claim 43, wherein the means for outputting the sleep apnea interruption pulse further comprises means for outputting the sleep apnea interruption pulse to at least one of the phrenic nerve and the diaphragm and to a portion of a heart to pace a rhythm of the heart.

47. The apparatus according to claim 37 wherein said means for determining if the patient is experiencing sleep apnea includes a detection device in wireless communication with an implanted medical device.
